(19) United States
(12) Patent Application Publication
(10) Pub. No.: US 2026/0283792 A1
(43) Pub. Date: Sep. 24, 2026

(54) PROSTHETIC VALVES INCLUDING FRAMES HAVING LATERAL PROTRUSIONS FOR COMMISSURE ATTACHMENT

(71) Applicant: Edwards Lifesciences Corporation, Irvine, CA (US)

(72) Inventor: Brendan Michael Dalbow, Oceanside, CA (US)

(73) Assignee: Edwards Lifesciences Corporation, Irvine, CA (US)

(21) Appl. No.: 19/654,735

(22) Filed: Apr. 22, 2026

Related U.S. Application Data
(63) Continuation of application No. PCT/US2024/052765, filed on Oct. 24, 2024.
Application No. PCT/US2024/052765 is a continuation of application No. 63/593,138, filed on Oct. 25, 2023.

(51) Int. Cl. A61F 2/24

(52) U.S. Cl. CPC A61F 2/2418; A61F 2/2415; A61F 2220/0075

(57) ABSTRACT

Prosthetic valves including an annular frame and a valvular structure mounted within the frame, and methods for assembling prosthetic valves. The valvular structure comprises a plurality of leaflets each including commissure tabs on opposing sides of the leaflet that can be paired with a commissure with an adjacent leaflet to form a commissure. The commissure is attached to a commissure support structure of the frame. The commissure support structure includes first and second axially extending struts defining a commissure window. Each of the first and second struts include laterally extending protrusions that extend outwardly relative to the commissure window. Each commissure tab can be attached to one of the struts via a primary suture looped around the lateral protrusions. A secondary suture can encircle the primary sutures and/or a flexible connector can be wrapped around the primary sutures to prevent dislodgement of the primary sutures from the lateral protrusions.

### CROSS-REFERENCE TO RELATED APPLICATIONS

This application is a continuation of PCT Application No. PCT/US2024/052765, filed Oct. 24, 2024, which claims the benefit of U.S. Provisional Patent Application No. 63/593,138, filed Oct. 25, 2023, the entire contents of each of which are incorporated herein by reference.

### FIELD

The present disclosure relates to prosthetic valves including frames that include commissure attachment structures, for example, commissure structures including one or more lateral protrusions, for coupling of leaflets to the frame.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic valves include a plurality of leaflets arranged in a leaflet assembly that cycles between closed and open states during the diastolic and systolic phases of the heart when implanted. The leaflets can each include commissure tabs on opposing sides of the leaflet that can be paired with a commissure tab on an adjacent leaflet. The paired commissure tabs can be attached to a commissure support structure in an outflow end portion of the frame to form commissures of the prosthetic valve.

### SUMMARY

Described herein are prosthetic heart valves, delivery apparatus, methods for implanting prosthetic heart valves, and methods for assembling prosthetic heart valves. The disclosed prosthetic heart valves and assembly methods can, for example, provide improved coupling and/or support structures for commissures of a leaflet assembly disposed within a frame of the prosthetic valve. As such, the devices and methods disclosed herein can, among other things, overcome one or more of the deficiencies of typical prosthetic heart valves and their delivery apparatus.

A prosthetic heart valve can comprise a frame and a leaflet assembly (which can also be referred to as a “plurality of leaflets” or a “valve structure” or a “valvular structure”) coupled to the frame. In addition to these components, a prosthetic heart valve can further comprise one or more of the components disclosed herein.

In some examples, a frame can include a plurality of angularly spaced commissure support structures for coupling of the commissure of the leaflet assembly thereto.

In some examples, a commissure support structure can include at least one laterally extending protrusion.

In some examples, a commissure support structure can include first and second axially extending struts that each have at least one laterally extending protrusion.

In some examples, a first commissure tab of a first leaflet can be connected to the first strut of the commissure support structure with a first suture wrapped around the at least one protrusion of the first strut, and a second commissure tab of a second leaflet can be connected to the second strut of the commissure support structure with a second suture wrapped around the at least one protrusion of the second strut.

In some examples, a commissure support structure can include first and second axially extending struts that each have a plurality of laterally extending protrusions.

In some examples, a commissure support structure can include gaps disposed between adjacent ones of the plurality of laterally extending protrusions.

In some examples, the first suture and the second suture can be woven through the respective commissure tabs at in regions corresponding to locations of the gaps between the laterally extending protrusions.

In some examples, the first and second axially extending struts can define a commissure window therebetween, and one or more laterally extending protrusions can extend from each of the first and second struts outwardly relative to the commissure window.

In some examples, a laterally extending protrusion can be perpendicular to relative to an exterior edge of the commissure window.

In some examples, a laterally extending protrusion can be perpendicular to relative to a longitudinal axis of the commissure window.

In some examples, a laterally extending protrusion can have a generally rectangular shape.

In some examples, a laterally extending protrusion can have a rectangular shape.

In some examples, a laterally extending protrusion can include an aperture.

In some examples, a laterally extending protrusion can have a length in a range of 0.3 mm to 1.5 mm.

In some examples, a laterally extending protrusion can have a length in a range of 0.6 to 0.9 mm.

In some examples, a flexible connector can extend over and/or cover at least a portion of the paired first and second commissure tabs.

In some examples, end portions of the flexible connector can each be coupled to an interior of the frame via one or more sutures.

In some examples, the flexible connector can wrap around the first and second sutures and side surfaces of the first and second struts.

In some examples, end portions of the flexible connector can be coupled to each other on an exterior of the frame via one or more sutures.

In some examples, the flexible connector can be configured to prevent dislodgment of the first suture from the laterally extending protrusion(s) of the first strut and dislodgement of the second suture from the laterally extending protrusion(s) of the second strut.

In some examples, the first and second sutures can be first and second primary, axially extending sutures.

In some examples, a secondary, laterally extending suture can extend from a first gap between adjacent laterally extending protrusions on the first strut to a second gap between adjacent laterally extending protrusions on the second strut.

In some examples, a secondary, laterally extending suture can extend from a first aperture in a laterally extending protrusion on the first strut to a second aperture in a laterally extending protrusion on the second strut.

In some examples, a secondary, laterally extending suture can be threaded through a first aperture in a laterally extending protrusion on the first strut and can be threaded through a second aperture in a laterally extending protrusion on the second strut.

In some examples, a secondary, laterally extending suture can extend around the first and second primary, axially extending sutures.

In some examples, a secondary, laterally extending suture can encircle the first and second primary, axially extending sutures.

In some examples, a secondary, laterally extending suture can be configured to prevent dislodgment of the first suture from the laterally extending protrusion(s) of the first strut and dislodgement of the second suture from the laterally extending protrusion(s) of the second strut.

In some examples, a prosthetic heart valve comprises a radially expandable and collapsible annular frame, the frame comprising a plurality of angularly spaced commissure support structures, each of the commissure support structures comprising axially oriented first and second struts, wherein each of the first and second struts comprises at least one laterally extending protrusion; and a leaflet assembly comprising a plurality of leaflets each comprising commissures tabs on opposite sides of the leaflet, wherein a first commissure tab of each leaflet is paired with a second commissure tab of an adjacent leaflet to form a commissure, wherein each commissure extends radially between the first and second struts of one of the commissure support structures, wherein the first commissure tab is connected to the first strut with a first suture wrapped around the at least one protrusion of the first strut and the second commissure tab is connected to the second strut with a second suture wrapped around the at least one protrusion of the second strut.

In some examples, a prosthetic heart valve comprises A prosthetic heart valve comprising: a radially expandable and collapsible annular frame, the frame comprising a commissure support structure including axially oriented first and second struts, wherein the first strut comprises a plurality of first laterally extending protrusions and the second strut comprises a plurality of second laterally extending protrusions; and a leaflet assembly comprising a plurality of leaflets disposed within the frame, wherein a first commissure tab of a first one of the leaflets is paired with a second commissure tab of a second, adjacent one of the leaflets to form a commissure, wherein the first commissure tab is coupled to the first laterally extending protrusions via a first suture, and wherein the second commissure tab is coupled to the second laterally extending protrusions via a second suture.

In some examples, a prosthetic heart valve comprises one or more of the components recited in Examples 1-29 below.

In some examples, a method of assembling a prosthetic heart valve can include inserting paired first and second commissure tabs between first and second axially extending struts of a commissure support structure of an annular frame, the commissure support structure comprising one or more first lateral protrusions extending from the first strut and one or more second lateral protrusions extending from the second strut.

In some examples, a method of assembling a prosthetic heart valve can include coupling the first commissure tab to the one or more first lateral protrusions via a first suture and coupling the second commissure tab to the one or more second lateral protrusions via a second suture.

In some examples, the first suture can be pre-attached to the first commissure tab and the second suture can be pre-attached to the second commissure tab prior to inserting the commissure tabs between the first and second axially extending struts.

In some examples, the coupling the first commissure tab can include looping one or more stitches of the first suture over the one or more first lateral protrusions and the coupling the second commissure tab can include looping one or more stitches of the second suture over the one or more second lateral protrusions.

In some examples, the coupling the first commissure tab can include aligning the first commissure tab with the one or more first lateral protrusions and stitching the first suture through the first commissure tab and around the one or more first lateral protrusions, and the coupling the second commissure tab can include aligning the second commissure tab with the one or more second lateral protrusions and stitching the second suture through the second commissure tab and around the one or more second lateral protrusions.

In some examples, the method can include stitching and/or forming a secondary, laterally extending suture around first and second primary, axially extending sutures.

In some examples, the secondary suture can be stitched through the commissure tab material at regions corresponding to gaps between laterally extending protrusions.

In some examples, the secondary suture can be stitched through the commissure tab material at regions corresponding to apertures in the laterally extending protrusions.

In some examples, the method can include covering at least a portion of the first and second commissure tabs with a flexible connector.

In some examples, the method can include wrapping a first portion the flexible connector over the first lateral protrusion(s) and the first suture, and wrapping a second portion of the flexible connector over the second lateral protrusion(s) and the second suture.

In some examples, the method can include coupling first and second end portions of the flexible connector to each other exterior of an exterior surface of the frame.

In some examples, the method can include sterilizing the assembled prosthetic heart valve.

In some examples, a method comprises one or more of the steps and/or elements recited in Examples 30-39 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of an exemplary prosthetic heart valve.

FIG. 2 is a side view of an example of a delivery apparatus configured to deliver and implant a radially expandable prosthetic heart valve at an implantation site.

FIG. 3 is a perspective view of another exemplary a prosthetic heart valve.

FIG. 4 is an elevation view of an exemplary commissure support structure including a plurality of laterally extending protrusions, according to one example.

FIGS. 5 and 6 are schematic illustrations of top views of exemplary commissure configurations for the commissure support structure of FIG. 4.

FIGS. 7 and 8 are schematic illustrations of front views of exemplary commissure configurations for an exemplary commissure support structure including a plurality of laterally extending protrusions.

FIG. 9 is a schematic illustration of a front view of an exemplary commissure configuration for an exemplary commissure support structure including a plurality of laterally extending protrusions.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example” and “i.e.” means “that is.”

### Overview of the Disclosed Technology

As introduced above, prosthetic valves can include a plurality of leaflets arranged in a leaflet assembly (which can also be referred to as a “valve structure,” and/or a “valvular structure”). The leaflets can each include commissure tabs on opposing sides of the leaflet that can be paired with a commissure tab on an adjacent leaflet. The paired commissure tabs can be attached to the frame at a commissure support structure (for example, a commissure window) in an outflow end portion of the frame.

The commissure window can be formed by a pair of axially aligned struts. In some examples, the paired commissure tabs can be covered by a flexible connector that can function to protect the tabs from wear or weakening caused by contact with the struts. The commissure tabs and/or the flexible connector can be inserted through the commissure window and folded outwardly (for example, away from each other) over an exterior surface of the frame. The commissure tabs and/or the flexible connector can then be attached to the struts, for example, by suturing the commissure tabs to a separate tab or a separate section of leaflet material on an interior of the frame, to secure the commissure tabs to the frame and form a commissure of the prosthetic valve. Thus, in many known prosthetic valves, attachment of the commissures can be complex and/or time-consuming during assembly of the valve.

Accordingly, there is a need for frames and methods of attachment that simplify coupling of commissure tabs to a frame of a prosthetic valve when forming commissures.

The prosthetic valves disclosed herein can address one or more of the foregoing issues of known prosthetic valves. In some examples, a prosthetic valve in accordance with the present disclosure can include a frame having angularly spaced commissure support structures that each include first and second axially aligned struts, wherein each of the struts has one or more protrusions (which can also be referred to as “extensions,” “projections,” and/or “fingers”) that laterally extend from a side edge of the strut, such as an edge of the strut forming an exterior edge of the commissure window. In some examples, paired commissure tabs of adjacent leaflets can be inserted between the first and second struts (for example, through the commissure window) and folded outward over the exterior surface of the frame. A suture (for example, a primary suture) can be threaded through and/or over one of the commissure tabs and wrapped around one or more lateral protrusions of the strut to secure the respective commissure tab to the frame. In some examples, a suture (for example, a primary suture) can be threaded through and/or over one of the commissure tabs and further threaded through apertures in one or more lateral protrusions of the strut to secure the respective commissure tab to the frame.

In some examples, struts of the commissure support structure can include a plurality of protrusions (for example, two, three, four or more protrusions), and, for each commissure tab, a suture can be woven through the commissure tab and between the plurality of protrusions, thereby forming two axially aligned or extending primary sutures. In some examples, one or more secondary sutures can be threaded through and/or around the commissure tabs and around the two primary, axially aligned sutures on opposing sides of the commissure support structure. In some examples, the secondary sutures extend through the commissure tabs and around the two primary sutures at gaps between the lateral protrusions on opposing sides of the commissure support structure. In some examples, the secondary sutures extend through the commissure tabs, around the two primary sutures at gap, and extend through apertures in the lateral protrusions on opposing sides of the commissure support structure. In some examples, a flexible connector can extend over (for example, cover) at least a portion of the paired commissure tabs, and the sutures can additionally be threaded through and/or over the flexible connector.

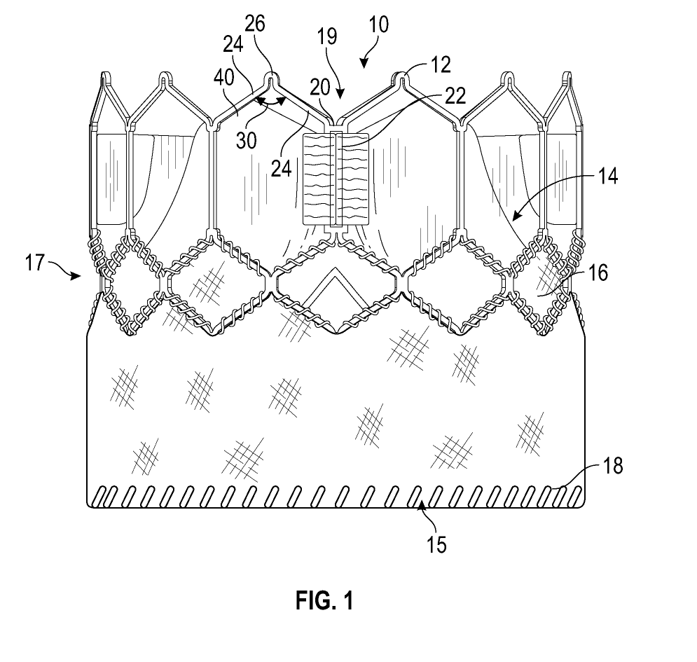

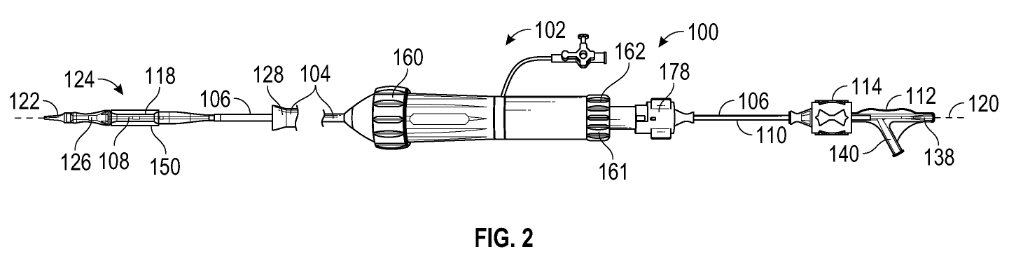

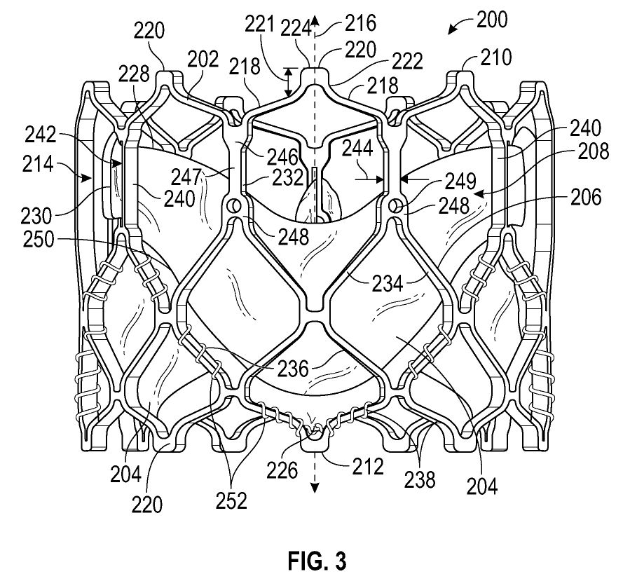

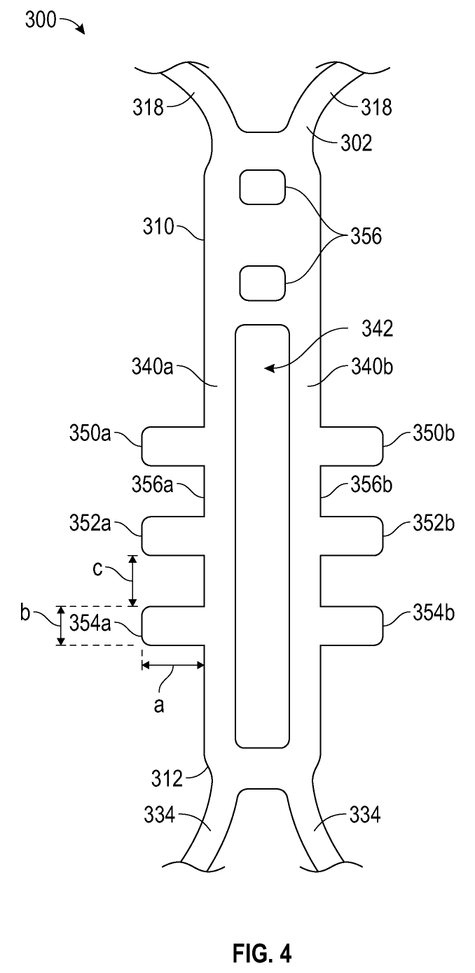

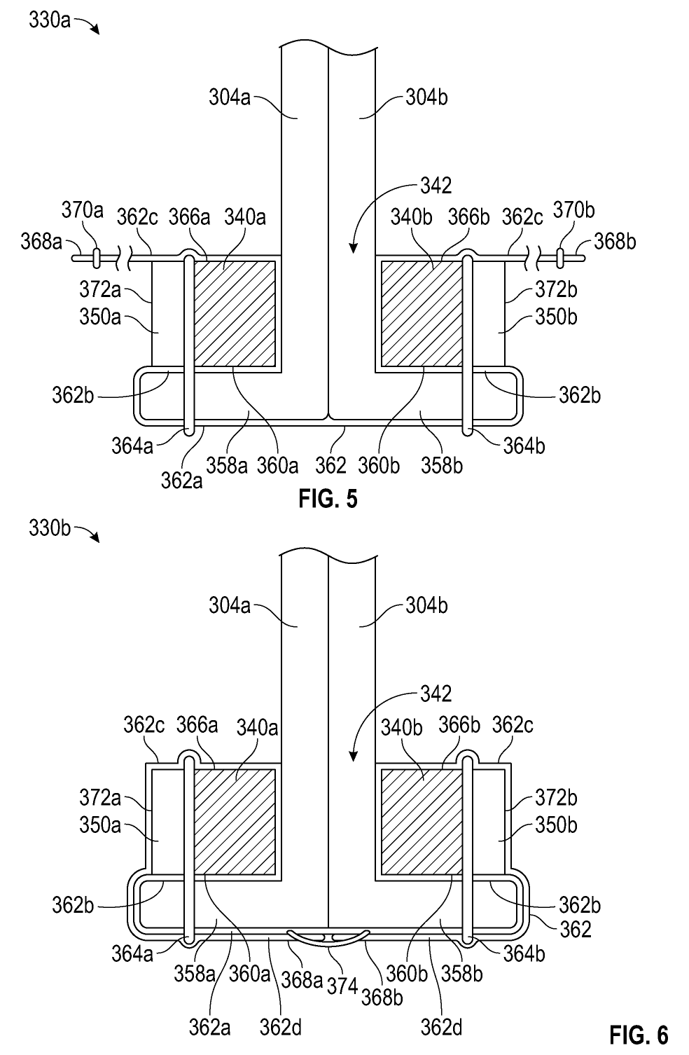

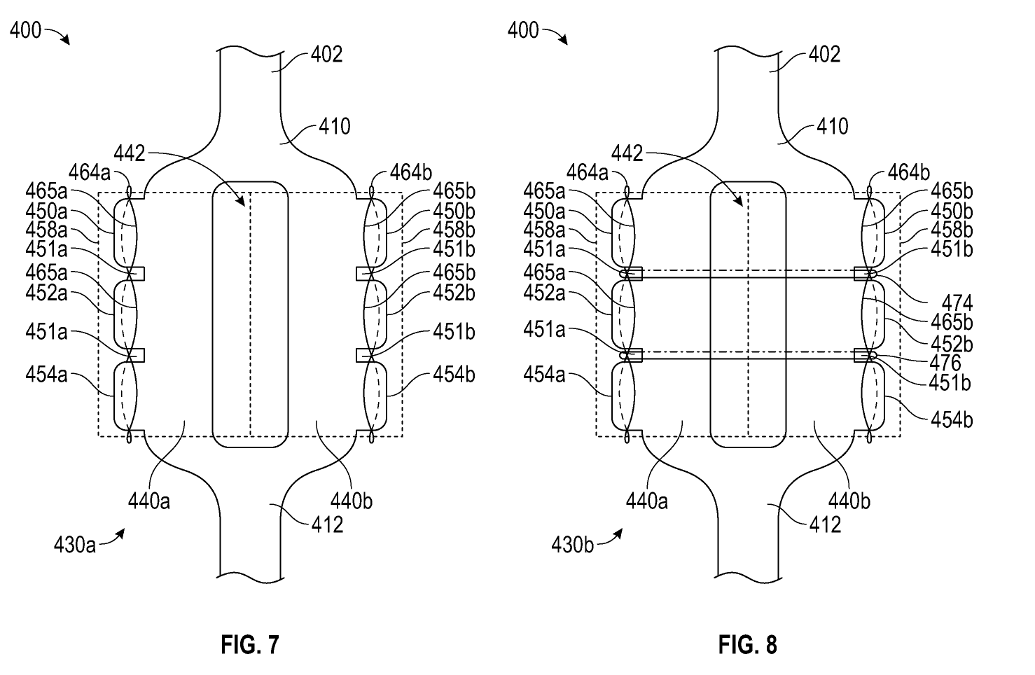

FIGS. 1 and 3 illustrate exemplary prosthetic valves. FIG. 2 illustrates an exemplary delivery apparatus that can be utilized the with prosthetic valves. FIG. 4 illustrates an exemplary commissure support structure, which includes laterally extending protrusions, for a frame of a prosthetic valve. FIGS. 5 and 6 illustrate exemplary suture configurations for coupling of the commissure tabs to the commissure support structure of FIG. 4. FIGS. 7 and 8 illustrate another exemplary commissure support structure including laterally extending protrusions.

The prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

### Examples of the Disclosed Technology

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Patent No. 10,363,130, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Patent No. 11,291,540, which is incorporated herein by reference.

The prosthetic valve 10 can have four main components: a stent or frame 12 (such as, for example, a radially expandable and collapsible annular frame), a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12 while the outer skirt 18 can be arranged on and/or coupled to an outer surface of the frame 12.

The inner and/or outer skirts can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the valvular structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein.

The frame 12 can be radially compressible (collapsible) and expandable (for example, expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially and/or angularly spaced slots, or commissure support structures 20 (which can also be referred to as “commissure windows”) that are adapted to mount the commissures 22 of the valvular structure 14 to the frame.

The frame 12 can be made of any of various suitable plastically-expandable materials (for example, stainless steel, etc.) or self-expanding materials (for example, Nitinol) as known in the art. When constructed of a plastically-expandable material, the frame 12 (and thus the valve 10) can be crimped to a radially compressed state on a delivery catheter and then expanded inside a patient by an inflatable balloon or equivalent expansion mechanism. When constructed of a self-expandable material, the frame 12 (and thus the valve 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

FIG. 2 shows an example of a delivery apparatus 100 that can be used to implant an expandable prosthetic heart valve (for example, prosthetic heart valve 10 of FIG. 1 or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient’s body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIGS. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient’s vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Patent No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Publication Nos. WO 2022/046591, WO/2022/046585, WO/2022/046588, which are each incorporated by reference herein.

FIG. 3 shows an example of a prosthetic heart valve 200 comprising a radially expandable and compressible annular frame 202 and a plurality of leaflets 204 secured to the frame (forming a leaflet assembly or valve structure). Each leaflet 204 can comprise opposing commissure tabs disposed on opposite sides of the leaflet 204 and a cusp edge portion extending between the opposing commissure tabs. Further, the leaflets can include an inflow end 226 and an outflow end 228. The leaflets 204 can be made from in whole or part, biological material, bio-compatible synthetic materials, or other such materials. Suitable biological material can include, for example, bovine pericardium (or pericardium from other sources). The leaflets 204 can be secured to one another at their adjacent sides to form commissures 230, each of which can be secured to a respective commissure support structure (also referred to herein as “commissure supports”) and/or to other portions of the frame, as described in greater detail below.

The frame 202 can be made of any of various suitable plastically-expandable materials (for example, stainless steel, etc.) or self-expanding materials (for example, nickel titanium alloy (NiTi), such as nitinol), as known in the art. In some examples, the frame 202 comprises a plastically-expandable material, such as those described above with reference to the prosthetic heart valve 10 of FIG. 1.

The frame 202 can comprise a plurality of interconnected struts 206 which form multiple rows of open cells 208 between an outflow end 210 and an inflow end 212 of the frame 202. In some examples, as shown in FIG. 3, the frame 202 can comprise three rows of cells 208 with a first (for example, upper in FIG. 3) row of cells 214, disposed at the outflow end 210, having cells 208 that are elongated in an axial direction (relative to a central longitudinal axis 216 of the frame 202), as compared to cells 208 in the remaining rows of cells. For example, the cells 208 of the first row of cells 214 can have a longer axial length, defined in a direction of a central longitudinal axis 216 of the frame 202, than cells 208 in the remaining rows of cells (for example, cells in the row of cells at the inflow end 212).

In some examples, as shown in FIG. 3, each row of cells 208 comprises nine cells. Thus, in such examples, the frame 202 can be referred to as a nine-cell frame.

In other examples, the frame 202 can comprise more than three rows of cells (for example, four or five) and/or more or less than nine cells per row. In some examples, the cells 208 in the first row of cells 214 may not be elongated compared to cells 208 in remaining rows of cells of the frame 202.

The interconnected struts 206 can include a plurality of angled struts 218, 234, 236, and 238 arranged in a plurality of rows of circumferentially extending rows of angled struts, with the rows being arrayed along the length of the frame between the outflow end 210 and the inflow end 212 of the frame 202. For example, the frame 202 can comprise a first row of angled struts 238 arranged end-to-end and extending circumferentially at the inflow end 212 of the frame; a second row of circumferentially extending, angled struts 236; a third row of circumferentially extending, angled struts 234; and a fourth row of circumferentially extending, angled struts 218 at the outflow end 210 of the frame 202. The fourth row of angled struts 218 can be connected to the third row of angled struts 234 by a plurality of axially extending window strut portions 240 and a plurality of axial (for example, axially extending) struts 232. The axially extending window strut portions 240 define commissure windows (for example, open windows) 242 that are spaced apart from one another around the frame 202, in a circumferential arrangement, and which are adapted to receive a pair of commissure tabs of a pair of adjacent leaflets 204 formed into a commissure 230.

One or more (for example, two, as shown in FIG. 3) axial struts 232 can be positioned between, in the circumferential direction, two commissure windows 242 formed by the window strut portions 240. Since the frame 202 can include fewer cells per row (for example, nine) and fewer axial struts 232 between each commissure window 242, as compared to other prosthetic heart valves, such as the prosthetic heart valve 10 of FIG. 1, each cell 208 can have an increased width (in the circumferential direction), thereby providing a larger opening for blood flow and/or coronary access.

Each axial strut 232 and each window strut portion 240 extends from a location defined by the convergence of the lower ends (for example, ends arranged inward of and farthest away from the outflow end 210) of two angled struts 218 (which can also be referred to as an upper strut junction or upper elongated strut junction) to another location defined by the convergence of the upper ends (for example, ends arranged closer to the outflow end 210) of two angled struts 234 (which can also be referred to as a lower strut junction or lower elongate strut junction). Each axial strut 232 and each window strut portion 240 forms an axial side of two adjacent cells of the first row of cells 214.

In some examples, as shown in FIG. 3, each axial strut 232 can have a width 244 that is larger than a width of the angled struts 218, 234, 236, and/or 238. As used herein, a “width” of a strut is measured between opposing locations on opposing surfaces of a strut that extend between the radially facing inner and outer surfaces of the strut (relative to the central longitudinal axis 216 of the frame 202). A “thickness” of a strut is measured between opposing locations on the radially facing inner and outer surfaces of a strut and is perpendicular to the width of the strut.

Commissure tabs of adjacent leaflets 204 can be secured together to form commissures 230. Each commissure 230 of the prosthetic heart valve 200 comprises two commissure tabs paired together, one from each of two adjacent leaflets 204, and extending through a commissure window 242 of the frame 202. Each commissure 230 can be secured to the window strut portions 240 forming the commissure window 242. Further details regarding the attachment of the commissures 230 to the windows 242 are described in U.S. 9,393,110, which is incorporated herein by reference.

The cusp edge portion (for example, scallop edge) of each leaflet 204 can be secured to the frame via one or more fasteners (for example, sutures). In some examples, as shown in FIG. 3, the cusp edge portion of each leaflet 204 can be secured directly to the struts of the frame 202 (for example, angled struts 234, 236, and 238). For example, the cusp edge portions 250 of the leaflets 204 can be connected to the angled struts 234, 236, 238 that generally follow the contour of the cusp edge portions of the leaflets via sutures 252.

In some examples, the cusp edge portions 250 of the leaflets 204 can be secured to an inner skirt and the inner skirt can then be secured directly to the frame 202.

Further, in some examples, an outer skirt can be connected to an outer surface of the frame 202 (for example, similar to the outer skirt 18 of the valve 10 of FIG. 1).

As illustrated in FIG. 3, in some examples, one or more of or each of the axial struts 232 can comprise an inflow end portion (for example, inflow end portion that is closer to the inflow end than the outflow end portion 246) 248 that is widened relative to a middle portion 247 of the axial strut 232 (which can be defined by the width 244), similar to the outflow end portion 246 (as described above). In some examples, the inflow end portion 248 of the axial strut 232 can comprise an aperture 249. The apertures 249 can be configured to receive fasteners (for example, sutures) for attaching soft components of the prosthetic heart valve 200 to the frame 202. For example, in some examples, an outer skirt can be positioned around an outer surface of the frame 202 and secured to the apertures 249 and/or other components of the frame.

The frame 202 can further comprise a plurality of apices 220 formed at the inflow end 212 and the outflow end 210, each apex 220 forming a junction between two angled struts 218 at the inflow end 212 or outflow end 210. As such, the apices 220 are spaced apart from one another, in a circumferential direction at the inflow end 212 and the outflow end 210. As shown in FIG. 3, each apex 220 can have side portions 222 that curve or bend axially outward from the angled strut 218 to which it is connected and an end portion 224 that extends between the two side portions 222 of the apex 220. The side portions 222 can extend in a direction that is parallel to the central longitudinal axis 216. The end portion 224 can be relatively flat and include a surface that is disposed normal to the central longitudinal axis 216. Each apex 220 can have two bends at its end portion 224 and two bends at the side portions 222 (for example, one at the junction between each side portion 222 and angled strut 218). In this way, the apices 220 can be U-shaped, similar to the apices 26 of the valve of FIG. 1.

Turning to FIGS. 4-9, exemplary commissure support structures including one or more laterally extending protrusions for coupling of leaflet commissure tabs to a frame of a prosthetic valve are shown and described. It will be appreciated that the exemplary commissure support structures and commissure configurations (for example, arrangements of commissure tabs, flexible connectors and/or sutures) can be incorporated into one or more of the prosthetic valves 10 or 200 discussed above, and/or other prosthetic valves. Further, one or more of the commissures and commissure support structures shown in FIGS. 4-9 can have one or more of the features of the commissure and commissure support structure features discussed above with respect to the prosthetic valves 10 or 200.

For example, FIG. 4 shows an exemplary commissure support structure 300 formed in a frame 302, which can have one or more features of the frames 12 or 202 discussed above and/or other features. As can be seen in FIG. 4, the commissure support structure 300 includes first and second axially extending struts 340a, 340b. In some examples, the first and second axially extending struts 340a, 340b can be connected at an outflow end portion 310 and an inflow end 312 portion of the commissure support structure 300, thereby defining a commissure window 342 therebetween. The commissure support structure 300 can be connected to angled struts 318, 334 of the frame 302, which, in some examples, can be similar to the angled struts 218, 234 discussed above. In other examples, the angled struts 318, 334 can differ from the angled struts 218, 234, such as, for example, having a different location within a frame and/or a different angle or shape relative to the angled struts 218, 234.

In some examples, the commissure support structure can additionally include apertures 356 disposed in the outflow end portion 310 of the commissure support structure 300 for receiving radiopaque markers, and/or for suturing a commissure to the commissure support structure 300. In some examples, the apertures 356 can be excluded.

Also shown in FIG. 4, each of the axially extending struts 340a, 340b can include a plurality of laterally extending protrusions (which can be referred to herein as “lateral protrusions”). In the present example, the axially extending strut 340a includes three laterally extending protrusions 350a, 352a, 354a, and the axially extending strut 340b includes three laterally extending protrusions 350b, 352b, 354b. In some examples, a commissure support structure can include fewer (for example, one or two) laterally extending protrusions. In some examples, a commissure support structure can include more (for example, four, five, six or more) laterally extending protrusions. In some examples, a number of protrusions on one side of the commissure window can differ relative to a number of protrusions on the second side of the commissure window.

In some examples, as shown in FIG. 4, each of the protrusions 350a, 352a, 354a, 350b, 352b, 354b can extend laterally away from (that is, outwardly relative to) the commissure window 342. In some examples, each of the protrusions 350a, 352a, 354a, 350b, 352b, 354b can be perpendicular to an exterior side edge 356a, 356b (forming exterior edges of the commissure window 342). In some examples, one or more of the protrusions 350a, 352a, 354a, 350b, 352b, 354b can be disposed at an angle relative to the exterior side edge 356a, 356b. For example, selected ones of the protrusions 350a, 352a, 354a, 350b, 352b, 354b can be angled toward either the outflow end 310 or the inflow end 312 of the commissure support structure 300.

In some examples, as shown in FIG. 4, each of the protrusions on the (first) strut 340a (on a first side of the commissure window) can be laterally aligned with an opposing protrusion on the (second) strut 340b (on a second side of the commissure window). For example, the protrusion 350a can be laterally aligned with the protrusion 350b, the protrusion 352a can be laterally aligned with the protrusion 352b, and the protrusion 354a can be laterally aligned with the protrusion 354b. In some examples, the opposing protrusions can be offset relative to each other in the axial direction.

In some examples, as shown in FIG. 4, each of the protrusions 350a, 352a, 354a, 350b, 352b, 354b can have a rectangular shape or a generally rectangular shape (for example, a rectangle including curved corner edges at a distal end of the protrusion). In some examples, one or more of the protrusions 350a, 352a, 354a, 350b, 352b, 354b can have a different shape. For example, the protrusions can be curved, such as being curved toward one of the outflow end 310 or the inflow end 312 of the commissure support structure 300. In another example, the protrusions can have a tapered shape that narrows towards the respective exterior side edge 356a, 356b. In another example, the protrusions can include a raised lip and/or a wider end portion opposing the exterior side edge 356a, 356b.

In some examples, as can be seen in FIG. 4, each of the protrusions can have a length a, which can be equal to a distance that the protrusion protrudes relative to the respective exterior side edge 356a, 356b. Further, each protrusion can have a height b, and each of the protrusions can have a distance c between the protrusion and an adjacent protrusion. In some examples, the length a can be in a range of 0.3 mm to 1.5 mm, such as, for example, 0.4 mm to 1.0 mm, 0.5 mm to 0.9 mm, 0.6 mm to 0.8 mm. In some examples, the height b is in a range of 0.3 mm to 1.5 mm, such as, for example, 0.4 mm to 1.0 mm, 0.5 mm to 0.9 mm, 0.6 mm to 0.8 mm. In some examples, the distance c can be in a range of 0.1 mm to 0.8 mm, such as, for example 0.2 mm to 0.6 mm, 0.3 mm to 0.55 mm, 0.4 to 0.45 mm. For example, length a can be 0.8 mm, the height b can be 0.8 mm, and the distance c can be 0.45 mm.

It will be appreciated that in other examples the protrusions can have other dimensions, which can be selected, for example, based on one or more other dimensions or features of the frame and/or the leaflets of the prosthetic valve. For example, for a larger prosthetic valve, such as a valve configured for implantation in a native mitral valve, the dimensions of the projections can be larger relative to a smaller prosthetic valve, such as a valve configured for implantation in a native aortic valve. In another example, dimensions of the protrusions can be selected based on a material of the leaflets. In another example, dimensions of the protrusions can be selected based on whether a flexible connector is utilized to cover the commissure tabs of the leaflets. In yet another example, dimensions of the protrusions can be selected based on a material of and/or a width of a suture material and/or a number of sutures used to connect the commissure tabs to the protrusions. In some examples, one or more of the protrusions can have different dimensions relative to others of the protrusions.

In some examples, a commissure can be formed at the commissure support structure 300 by pairing two commissure tabs of two adjacent leaflets, extending them through the commissure window 342, and folding and/or wrapping the leaflets outwardly (away from each other) over the exterior surface of the struts 340a, 340b. The leaflets can the be attached to respective ones of the protrusions 350a, 352a, 354a and 350b, 352b, 354b via sutures. The leaflets can optionally be covered by a flexible connector (which can also be referred to as “a cover,” “a protective sheet,” “a reinforcement layer”). FIGS. 5 and 6 show schematic illustrations of exemplary commissure configurations for forming commissures 330a, 330b at the commissure support structure 300.

Specifically, FIG. 5 illustrates a commissure 330a, according to one example, and FIG. 6 illustrates a commissure 330b, according to another example. In some examples, the commissures 330a, 330b can have one or more of the features of the commissures 22 and/or 230, discussed above and illustrated in FIGS. 1 and 3. In some examples, the commissures 330a, 330b can have one or more of the features that differ from the commissures 22 and/or 230.

As can be seen in FIGS. 5 and 6, the commissures 330a, 330b can each include adjacent leaflets 304a, 304b that are paired at their respective commissure tabs 358a, 358b. The paired commissure tabs 358a, 358b can be inserted between the axially extending struts 340a, 340b and through the commissure window 342. Further, the commissure tabs 358a, 358b can be folded outwardly (for example, away from each other) over respective exterior surfaces 360a, 360b of the struts 340a, 340b.

In some examples, a flexible connector 362 extends over (for example, covers) surfaces of the paired commissure tabs 358a, 358b that would otherwise contact the struts 340a, 340b. In the example of FIG. 5, the flexible connector 362 is folded to form a first layer 362a that extends across the exterior surfaces of the commissure tabs 358a, 358b. The first layer 362a is folded around the outer edges of the commissure tabs 358a, 358b to form respective second layers 362b that extend between the commissure tabs 358a, 358b and the exterior surfaces 360a, 360b of the struts 340a, 340b. Each second layer 362b extends through the commissure window and wraps around a respective strut 340a, 340b to form a respective third layer 362c that extends across an interior surface 366a, 366b of a respective strut 340a, 340b. In some examples, the flexible connector 362 can protect and/or prevent wear on the commissure tabs 358a, 358b that can be caused by contact with the struts 340a, 340b. In some examples, a flexible connector can cover only a portion of the paired commissure tabs. In some examples, a flexible connector can be excluded.

Each of the commissure tabs 358a, 358b can be coupled to a respective one of the struts 340a, 340b by a primary suture 364a, 364b. As shown in FIGS. 5 and 6, the primary sutures 364a, 364b can wrap over a respective one of the commissure tabs 358a, 358b (including a portion of the flexible connector 362 covering the commissure tab 358a, 358b) and over a respective one of the protrusions 350a, 350b, and extend to an interior surface 366a, 366b of the struts 340a, 340b.

In the exemplary commissure 330a shown in FIG. 5, end portions 368a, 368b of the flexible connector 362 can extend from the interior surfaces 366a, 366b of the struts 340a, 340b to another strut (not shown) of the frame 302, such as, for example, an adjacent strut. The end portions 368a, 368b can be coupled to interior surface of a strut via sutures 370a, 370b.

Different from the example shown in FIG. 5, in the commissure 330b of FIG. 6, the flexible connector 362 can be wrapped from the interior surface 366a, 366b around side surfaces 372a, 372b and over the outwardly folded commissure tabs 358a, 358b and the first layer 362a, thereby forming fourth layers 362d of the flexible connector 362 covering the primary sutures 364a, 364b. The end portions 368a, 368b can be attached to each other via a suture 374 exterior of an exterior surface 360a, 360b of the struts 340a, 340b. In some examples, the configuration of the flexible connector 362 in the commissure 330b (that is, a configuration where a flexible connector 362 is wrapped from the interior surface 366a, 366b around side surfaces 372a, 372b and over the outwardly folded commissure tabs 358a, 358b) can assist in retaining a position of the primary sutures 364a, 364b relative to the struts 340a, 340b (for example, by preventing the primary sutures from slipping outwardly over and/or dislodging from the protrusions 350a, 350b, as well as other protrusions of the commissure support structure 300).

In some examples, although not visible in the top views of FIGS. 5 and 6, the primary sutures 364a, 364b can extend through the commissure tabs 358a, 358b and layers 362a, 362b of the flexible connector 362, and can be wrapped around and/or woven between others of the protrusions (for example, the protrusions 352a, 354a and 352b, 354b). In some examples, the primary sutures 364a, 364b can be interwoven through the commissure tabs 358a, 358b and one or more layers of the flexible connector 362 and between the protrusions 350a, 352a, 354a and 350b, 352b, 354b (similar to the examples shown in FIGS. 7 and 8). In some examples, the primary sutures 364a, 364b can have a different configuration at one or more others of the protrusions.

It will be appreciated that the foregoing commissure configurations are merely exemplary, and the commissure support structure 300 can have other commissure configurations. For example, the commissures 330a, 330b can exclude the flexible connector 362. In another example, the commissures 330a, 330b can include additional primary sutures and/or one or more secondary sutures (for example, secondary sutures similar to those discussed below with reference to FIG. 8).

Turning to FIGS. 7 and 8, another exemplary commissure support structure 400 in a frame 402 is shown and described. The commissure support structure 400 can have one or more of the features of the commissure support structure 300 discussed above. For example, as can be seen in FIGS. 7 and 8, the commissure support structure 400 can include first and second axially extending struts 440a, 440b connected at an outflow end portion 410 and an inflow end portion 412 of the commissure structure 400 to define a commissure window 442 therebetween. Further, each of the struts 440a, 440b can respectively include laterally extending protrusions 450a, 452a, 454a and 450b, 452b, 454b.

Paired and outwardly folded commissure tabs 458a, 458b that extend through the commissure window 442 to form commissures 430a, 430b are schematically illustrated in dashed line in each of FIGS. 7 and 8. Although not shown, the commissures 430a, 430b can additionally include a flexible connector 362 having a configuration similar to those shown in FIGS. 5 or 6, or a different configuration.

In the present example, primary sutures 464a, 464b can be axially arranged and/or axially extending sutures, each including a plurality of stitches 465a, 465b. The sutures 464a, 464b are schematically illustrated in FIGS. 7 and 8, and non-visible portions (that is, portions behind the lateral protrusions) of the sutures 464a, 464b are shown in dashed line (a larger dashed line relative to the dashed line of the commissure tabs 458a, 458b).

In some examples, the primary sutures 464a, 464b can be generally parallel to the axially extending struts 440a, 440b. In some examples, a respective one of the primary sutures 464a, 464b can be woven through the commissure tabs 458a, 458b (an optionally through layers 362a, 362b of the connector 362 if a connector is present) at gaps 451a, 451b between the laterally extending protrusions 450a, 452a, 454a and 450b, 452b, 454b, and can be further woven through the commissure tabs 458a, 458b (an optionally through layers 362a, 362b of the connector 362 if a connector is present) at regions (or locations) above the protrusion 450a, 450b and below the protrusion 454a, 454b, as shown in FIGS. 7 and 8. In such examples, the primary sutures 464a, 464b can each have four intersection points (or intersection regions) where the primary suture passes through the commissure tab 458a, 458b (and optionally the connector), and each suture can include three stitches 465a, 465b in the form of loops that extend around (for example, loop over and/or wrap around) the respective laterally extending protrusions 450a, 452a, 454a and 450b, 452b, 454b.

In some examples, each of the primary sutures 464a, 464b can extend over the inflow and outflow edges (upper and lower edges in FIGS. 7 and 8) of the commissure tab 458a, 458b and can exclude intersection points at the locations above the protrusion 450a, 450b and below the protrusion 454a, 454b. In such examples, the primary suture 464a, 464b can be woven through the commissure tabs 458a, 458b (and optionally the connector) at the gaps 451a, 451b between the laterally extending protrusions 450a, 452a, 454a and 450b, 452b, 454b. In such examples, the primary sutures 464a, 464b can each have two intersection points (or intersection regions) where the suture passes through the respective commissure tab 458a, 458b, and can include three stitches 465a, 465b that extend around (for example, loop over) the respective laterally extending protrusions 450a, 452a, 454a and 450b, 452b, 454b.

As discussed above, in some examples, a commissure support structure can include fewer (for example, two) laterally extending protrusions or more (for example, four or five) laterally extending protrusions. It will be appreciated that a number of intersection regions for a primary suture can vary depending on the number of laterally extending protrusions included the commissure support structure.

As can be seen in the commissure 430b of FIG. 8, in some examples, secondary laterally extending sutures 474, 476 can extend around the primary sutures 464a and 464b. The secondary sutures 474, 476 are schematically illustrated in FIGS. 7 and 8, and non-visible portions (that is, portions behind the commissure support structure 400 and the commissure tabs 458a, 458b) of the sutures 474, 476 are shown in uneven broken line.

In some examples, the secondary suture 474 can extend around a portion (for example, an intersection region) of the primary suture 464a at the gap 451a between the protrusions 450a and 452a, and around a portion (for example, an intersection region) of the primary suture 464b at the gap 451b between the protrusions 450b and 452b. In some examples, the secondary suture 476 can extend around a portion (for example, an intersection region) of the primary suture 464a at the gap 451a between the protrusions 452a and 454a, and around a portion (for example, an intersection region) of the primary suture 464b at the gap 451b between the protrusions 452b and 454b.

Thus, in some examples, the secondary sutures 474, 476 can capture and/or encircle the primary sutures 464a, 464b. For example, the secondary sutures 474, 476 can capture and/or encircle the primary sutures 464a, 464b at the intersection regions within the gaps 451a, 451b between the protrusions on opposing sides of the commissure support structure 400. In some examples, the secondary sutures 474, 476 can retain positions of the primary sutures 464a, 464b relative to the struts 440a, 440b (for example, by preventing outward slipping over the laterally extending protrusions and/or dislodgement of the primary sutures from the laterally extending protrusions).

In some examples, the secondary sutures 474, 476 can couple the commissure tabs 458a, 458b to the struts 440a, 440b. In some examples, the primary sutures used in combination with secondary, lateral sutures can have a different configuration. For example, individual primary sutures can loop or wrap around a single protrusion rather than being woven through the gaps between the protrusions, and the secondary sutures can help to secure the commissure tabs to the frame without encircling the primary sutures. In some examples, the primary sutures can be excluded, and the laterally extending sutures can be used alone or in combination with other coupling mechanisms.

In some examples (as shown in FIG. 8), the laterally extending sutures 474, 476 can extend through the commissure tabs 458a, 458b (and optionally the connector 362). In some examples, the laterally extending sutures 474, 476 can wrap around edges of the commissure tabs 458a, 458b without penetrating the commissure tab material.

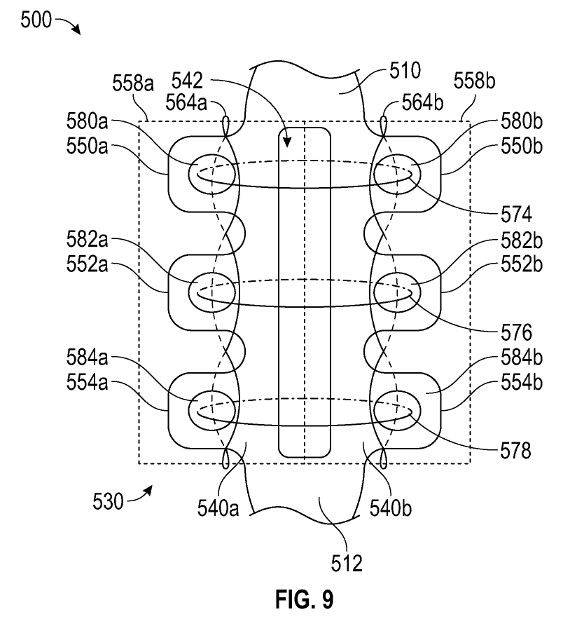

FIG. 9 illustrates another exemplary commissure support structure 500. The commissure support structure 500 can have one or more of the features of the commissure support structures 300, 400 discussed above. For example, as can be seen in FIG. 9, the commissure support structure 500 can include first and second axially extending struts 540a, 540b connected at an outflow end portion 510 and an inflow end portion 512 of the commissure structure 500 to define a commissure window 542 therebetween. Further, each of the struts 540a, 540b can respectively include laterally extending protrusions 550a, 552a, 554a and 550b, 552b, 554b.

Paired and outwardly folded commissure tabs 558a, 558b that extend through the commissure window 542 to form a commissure 530 are schematically illustrated in dashed lines. Although not shown, the commissure 530 can additionally include a flexible connector 362 having a configuration similar to the those shown in FIGS. 5 or 6, or a different configuration.

The primary sutures 564a, 564b can be axially arranged and/or axially extending sutures, and can have a configuration similar to that of the primary sutures 464a, 464b, described above. The sutures 564a, 564b are schematically illustrated in FIG. 9, and non-visible portions (that is, portions behind the lateral protrusions and/or the commissure tabs) of the sutures 564a, 564b are shown in dashed line (a larger dashed line relative to the dashed line of the commissure tabs 558a, 558b). In some examples, as illustrated in FIG. 9, the primary sutures 564a, 564b can be woven through gaps between the laterally extending protrusions 550a, 552a, 554a and 550b, 552b, 554b.

Different from other commissure support structures disclosed herein, the laterally extending protrusions 550a, 552a, 554a and 550b, 552b, 554b in the commissure support structure 500 can each include an aperture. For example, the laterally extending protrusions 550a, 552a, 554a and 550b, 552b, 554b can respectively include apertures 580a, 582a, 584a and 580b, 582b, 584b. In some examples, rather than being woven through the gaps between the laterally extending protrusions 550a, 552a, 554a and 550b, 552b, 554b (as shown in FIG. 9), the primary sutures 564a, 564b can be woven through the respective apertures 580a, 582a, 584a and 580b, 582b, 584b.

In the illustrated example shown in FIG. 9, in some examples, secondary laterally extending sutures 574, 576, 578 can extend around the primary sutures 564a and 564b. The secondary sutures 574, 576, 578 are schematically illustrated in FIG. 9, and non-visible portions (that is, portions behind the commissure support structure 500 and the commissure tabs 558a, 558b) of the sutures 574, 576, 578 are shown in uneven broken line.

In some examples, the secondary suture 574 can extend around portions of the primary sutures 564a, 564b and can be threaded through the apertures 580a, 580b in the opposing lateral protrusions 550a, 550b. In some examples, the secondary suture 576 can extend around portions of the primary sutures 564a, 564b and can be threaded through the apertures 582a, 582b in the opposing lateral protrusions 552a, 552b. In some examples, the secondary suture 578 can extend around portions of the primary sutures 564a, 564b and can be threaded through the apertures 584a, 584b in the opposing lateral protrusions 554a, 554b.

Thus, in some examples, the secondary sutures 574, 576, 578 can capture and/or encircle the primary sutures 564a, 564b. For example, the secondary sutures 574, 576, 578 can capture and/or encircle the primary sutures 564a, 564b at portions thereof that overlap with and/or extend over the laterally extending protrusions 550a, 552a, 554a and 550b, 552b, 554b. In some examples, the secondary sutures 574, 576, 578 can assist in retaining positions of the primary sutures 564a, 564b relative to the struts 540a, 540b (for example, by preventing outward slipping over the laterally extending protrusions and/or dislodgement of the primary sutures from the laterally extending protrusions).

In some examples, the secondary sutures 574, 576, 578 can couple the commissure tabs 558a, 558b to the struts 5440a, 540b. In some examples, the primary sutures used in combination with secondary, lateral sutures can have a different configuration. For example, individual primary sutures can each loop or wrap around a single protrusion rather than being woven through the gaps between the protrusions, and the secondary sutures 574, 576, 578 can encircle the induvial primary sutures on opposing sides of the commissure window. In another example, the primary sutures can be threaded through the apertures in the lateral protrusions to secure the respective commissure tab to the frame. In some examples, the primary sutures can be excluded, and the laterally extending sutures can be used alone or in combination with other coupling mechanisms.

In some examples (as shown in FIG. 9), the laterally extending sutures 574, 576, 578 can extend through the commissure tabs 558a, 558b (and optionally the connector 362). In some examples, the laterally extending sutures 574, 576, 578 can wrap around edges of the commissure tabs 558a, 558b without penetrating the commissure tab material (for example, where the commissure tabs 558a, 558b are shorter and do not extend past the lateral protrusions).

In some examples, a commissure can include one or more first secondary sutures that extend from a gap between first lateral protrusions to a gap between second lateral protrusions on an opposing side of the commissure (such as, for example, the secondary sutures 474, 476 of FIG. 8), and can further include one or more second secondary sutures that extend from an aperture in a first lateral protrusion to an aperture in a second lateral protrusions on an opposing side of the commissure (such as, for example, the secondary sutures 574, 576, 578 of FIG. 9).

In some examples, one or more of the sutures disclosed herein can comprise a high tensile strength biocompatible material, such as an ultra-high molecular weight polyethylene (UHMPE) force fiber®, polypropylene (for example, Prolene 4-0), polytetrafluoroethylene (PTFE), expanded PTFE (ePTFE), or other materials or combinations thereof.

In some examples, the flexible connector 362 can be made of any of the materials disclosed above in connection with the inner and outer skirts 16, 18. In one specific example, the flexible connector 262 can comprise a woven PET fabric.

### Exemplary Assembly Methods

In some examples, a method of forming a commissure of a prosthetic valve can include positioning leaflets inside of a frame for the prosthetic valve, and inserting a pair of commissure tabs of adjacent leaflets through a commissure window of a commissure support structure (such as, the commissure support structures shown in FIGS. 4-9). In some examples, the commissure window can be defined by first and second axially extending struts each having one or more laterally extending protrusions. In some examples, the method can further include outwardly folding the commissure tabs over an exterior surface of the struts and coupling the commissure tabs to the laterally extending protrusions. In some examples, the method can include covering the commissure tabs with a flexible connector prior to their insertion through the commissure window.

In some examples, the method can include suturing the commissures (and the flexible connector, if included) to the laterally extending protrusions, thereby forming first and second primary, axially arranged sutures (such as, the first and second primary sutures shown in FIGS. 5-9). In some examples, the primary sutures can be continuous sutures including a plurality of stitches. In some examples, the primary sutures can be a plurality of individual sutures each including one stitch. In some examples, the suturing can be performed with a blunt needle. The same procedure can be followed for mounting the other commissures to the frame. As can be appreciated, this technique can significantly simplify the valve assembly process by enabling suturing of the commissure tabs directly to the frame.

In some examples, rather than forming the primary sutures after positioning of the paired commissure tabs in the commissure support structure, first and second primary sutures (such as, the first and second primary sutures shown in FIGS. 5-9) can be pre-attached to the commissure tabs (and the flexible connector, if included) prior to placing the leaflets in the frame and inserting the commissure tabs through the commissure window. The stitches of the primary sutures (which can be continuous sutures including a plurality of stitches or a plurality of individual sutures each including one stitch) can then be slid over respective laterally extending protrusions (resulting in, for example, insertion of the laterally extending protrusions through the stitches) for coupling of the commissure tabs to the first and second axially extending struts. The same procedure can be followed for mounting the other commissures to the frame. As can be appreciated, this technique can significantly simplify the valve assembly process, as most stitching can be accomplished while the leaflets are outside of the frame.

In some examples, the method can include forming one or more secondary sutures that extend around the first and second primary sutures. In some examples, the secondary sutures can extend around the first primary at a first gap between protrusions of the first strut, and can further extend around the second primary suture at a second gap between protrusions on the second strut, wherein the first gap is on an opposing side of the commissure support structure relative to the second gap (for example, as shown in FIG. 8). In some examples, the lateral protrusions can each include an aperture, and a secondary suture can be threaded through apertures of lateral protrusions on opposing sides of the commissure window and can extend around the first and second primary sutures (for example, as shown FIG. 9). As can be appreciated, these techniques can assist in retaining positions of the first and second primary sutures relative to the commissure support structure and securing the commissure tabs to the frame by, for example, limiting or preventing outward slipping and/or dislodgement of the primary sutures from the lateral protrusions.

As noted above, in some examples, the method can include wrapping a flexible connector over the paired commissure tabs to, for example, protect the commissure tabs from contact with the axially extending struts. In some examples, the method can include coupling first and second end portions of the flexible connector to the frame on an interior of the frame.

In some examples, the method can include wrapping a first portion the flexible connector over the lateral protrusions of the first strut and the first primary suture, and wrapping a second portion of the flexible connector over lateral protrusions of the second strut and the second primary suture. In some examples, the method can include coupling first and second end portions of the flexible connector to each other exterior of an exterior surface of the frame. As can be appreciated, this technique can assist in retaining positions of the first and second primary sutures relative to the commissure support structure and securing the commissure tabs to the frame by, for example, limiting or preventing outward slipping and/or dislodgement of the primary sutures from the lateral protrusions.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a radially expandable and collapsible annular frame, the frame comprising a plurality of angularly spaced commissure support structures, each of the commissure support structures comprising axially oriented first and second struts, wherein each of the first and second struts comprises at least one laterally extending protrusion; and a leaflet assembly comprising a plurality of leaflets each comprising commissures tabs on opposite sides of the leaflet, wherein a first commissure tab of each leaflet is paired with a second commissure tab of an adjacent leaflet to form a commissure, wherein each commissure extends radially between the first and second struts of one of the plurality of commissure support structures, wherein the first commissure tab is connected to the first strut with a first suture wrapped around the at least one protrusion of the first strut and the second commissure tab is connected to the second strut with a second suture wrapped around the at least one protrusion of the second strut.

Example 2. The prosthetic heart valve of any example disclosed herein, particularly example 1, wherein the first and second struts define a commissure window therebetween having a respective one of the commissures extending therethrough, and wherein the at least one protrusion of the first strut and the at least one protrusion of the second strut extend laterally outwardly relative to the commissure window.

Example 3. The prosthetic heart valve of any example disclosed herein, particularly example 2, wherein the at least one protrusion of the first strut is perpendicular relative to a first exterior edge of the commissure window, and wherein the at least one protrusion of the first strut is perpendicular relative to a second, opposing exterior edge of the commissure window.

Example 4. The prosthetic heart valve of any example disclosed herein, particularly examples 2 or 3, wherein the at least one protrusion of the first strut is aligned with the at least one protrusion of the second strut.

Example 5. The prosthetic heart valve of any example disclosed herein, particularly examples 1-4, wherein each of the at least one protrusion of the first strut and the at least one protrusion of the second strut is rectangular in shape.

Example 6. The prosthetic heart valve of any example disclosed herein, particularly examples 1-5, wherein a distal end of each of the at least one protrusion of the first strut and the at least one protrusion of the second strut comprises curved corner edges.

Example 7. The prosthetic heart valve of any example disclosed herein, particularly examples 1-6, wherein each of the at least one protrusion of the first strut and the at least one protrusion of the second strut has a length in a range of 0.6 mm to 0.9 mm.

Example 8. The prosthetic heart valve of any example disclosed herein, particularly examples 1-7, further comprising a flexible connector extending over at least a portion of the first commissure tab and at least a portion of the second commissure tab, the flexible connector configured to prevent contact between the first commissure tab and the first strut and to prevent contact between the second commissure tab and the second strut.

Example 9. The prosthetic heart valve of any example disclosed herein, particularly example 8, wherein the first suture extends through the first commissure tab and the flexible connector, and wherein the second suture extending through the second commissure tab and the flexible connector.

Example 10. The prosthetic heart valve of any example disclosed herein, particularly examples 8 or 9, wherein end portions of the flexible connector are each coupled to an interior of the frame via one or more sutures.

Example 11. The prosthetic heart valve of any example disclosed herein, particularly examples 8 or 9, wherein the flexible connector wraps around side surfaces of the first and second struts and end portions of the flexible connector are coupled to each other on an exterior of the frame via one or more sutures.

Example 12. The prosthetic valve of any example disclosed herein, particularly example 11, wherein the flexible connector is configured to prevent dislodgment of the first suture from the at least one protrusion of the first strut and dislodgement of the second suture from the at least one protrusion of the second strut.

Example 13. The prosthetic heart valve of any example disclosed herein, particularly examples 1-12, wherein the at least one protrusion of the first strut comprises a plurality of first protrusions, and the at least one protrusion of the second strut comprises a plurality of second protrusions.

Example 14. The prosthetic heart valve of any example disclosed herein, particularly example 13, wherein adjacent ones of the first protrusions have first gaps disposed therebetween and adjacent ones of the second protrusions have second gaps disposed therebetween.

Example 15. The prosthetic heart valve of any example disclosed herein, particularly example 14, the first suture is woven through the first commissure tab and the first gaps, and wherein the second suture is woven through the second commissure tab and the second gaps.

Example 16. The prosthetic heart valve of any example disclosed herein, particularly example 15, wherein the first and second sutures are first and second primary, axially extending sutures.

Example 17. The prosthetic heart valve of any example disclosed herein, particularly example 16, further comprising a secondary, laterally extending suture extending from one of the first gaps to one of the second gaps and around the first and second primary, axially extending sutures.

Example 18. The prosthetic heart valve of any example disclosed herein, particularly examples 1-16, wherein each of the at least one protrusion of the first strut and the at least one protrusion of the second strut comprises an aperture, and a secondary, laterally extending suture is threaded through the apertures of the at least one protrusion of the first strut and the at least one protrusion of the second strut such that the secondary suture extends around the first and second sutures.

Example 19. The prosthetic heart valve of any example disclosed herein, particularly examples 17 or 18, where the secondary suture encircles portions of each of the first and second sutures.

Example 20. The prosthetic heart valve of any example disclosed herein, particularly examples 17-19, wherein the secondary suture is configured to prevent dislodgment of the first suture from the at least one protrusion of the first strut and prevent dislodgement of the second suture from the at least one protrusion of the second strut.

Example 21. A prosthetic heart valve comprising: a radially expandable and collapsible annular frame, the frame comprising a commissure support structure including axially oriented first and second struts, wherein the first strut comprises a plurality of first laterally extending protrusions and the second strut comprises a plurality of second laterally extending protrusions; and a leaflet assembly comprising a plurality of leaflets disposed within the frame, wherein a first commissure tab of a first one of the leaflets is paired with a second commissure tab of a second, adjacent one of the leaflets to form a commissure, wherein the first commissure tab is coupled to the first laterally extending protrusions via a first suture, and wherein the second commissure tab is coupled to the second laterally extending protrusions via a second suture.

Example 22. The prosthetic heart valve of any example disclosed herein, particularly example 21, wherein the first and second struts define a commissure window therebetween, and wherein the paired first and second commissure tabs extend through the commissure window.

Example 23. The prosthetic heart valve of any example disclosed herein, particularly examples 21 or 22, wherein the first suture is woven between the first laterally extending protrusions, and wherein the second suture is woven between the second laterally extending protrusions.

Example 24. The prosthetic heart valve of any example disclosed herein, particularly examples 21-23, wherein the first suture extends through the first commissure tab at first gaps between the first laterally extending protrusions, and wherein the second suture extends through the second commissure tab at second gaps between the second laterally extending protrusions.

Example 25. The prosthetic heart valve of any example disclosed herein, particularly example 24, wherein a lateral suture extends from one of the first gaps to one of the second gaps on an opposing side of the commissure support structure such that the lateral suture extends around the first and second sutures.

Example 26. The prosthetic heart valve of any example disclosed herein, particularly examples 21-24, wherein the first laterally extending protrusions comprise first apertures and the second laterally extending protrusion comprise second apertures, and wherein a lateral suture extends from one of the first apertures to one of the second aperture on an opposing side of the commissure support structure such that the lateral suture extends around the first and second sutures.

Example 27. The prosthetic heart valve of any example disclosed herein, particularly examples 21-26, wherein the first suture comprises a plurality of first stitches and the second suture comprises a plurality of second stitches, wherein each of the first stitches is looped over one of the first laterally extending protrusions and each of the second stitches is looped over one of the second laterally extending protrusions.

Example 28. The prosthetic heart valve of any example disclosed herein, particularly examples 21-27, further comprising a flexible connector covering at least a portion of the paired first and second commissure tabs; wherein a first portion of the flexible connector extends from an interior surface of the frame, over the first laterally extending protrusions and the first suture, to an exterior surface of the frame, and wherein a second portion of the flexible connector extends from the interior surface of the frame, over the second laterally extending protrusions and the second suture, to an exterior surface of the frame.

Example 29. The prosthetic heart valve of any example disclosed herein, particularly example 28, wherein first and second end portions of the flexible connector are coupled to each other via a suture that is exterior of the exterior surface of the frame.

Example 30. A method of assembling a prosthetic heart valve, the method comprising: pairing a first commissure tab of a first leaflet with a second commissure tab of a second, adjacent leaflet; inserting the paired first and second commissure tabs between first and second axially extending struts of a commissure support structure of an annular frame, the commissure support structure comprising one or more first lateral protrusions extending from the first strut and one or more second lateral protrusions extending from the second strut; and coupling the first commissure tab to the one or more first lateral protrusions via a first suture and coupling the second commissure tab to the one or more second lateral protrusions via a second suture.

Example 31. The method of any example disclosed herein, particularly example 30, wherein the first suture is pre-attached to the first commissure tab and the second suture is pre-attached to the second commissure tab prior to inserting the first and second commissure tabs between the first and second axially extending struts, and wherein the coupling the first commissure tab comprises looping one or more stitches of the first suture over the one or more first lateral protrusions and the coupling the second commissure tab comprises looping one or more stitches of the second suture over the one or more second lateral protrusions.

Example 32. The method of any example disclosed herein, particularly example 30, wherein the coupling the first commissure tab comprises aligning the first commissure tab with the one or more first lateral protrusions and stitching the first suture through the first commissure tab and around the one or more first lateral protrusions, and wherein the coupling the second commissure tab comprises aligning the second commissure tab with the one or more second lateral protrusions and stitching the second suture through the second commissure tab and around the one or more second lateral protrusions.

Example 33. The method of any example disclosed herein, particularly examples 30-32, wherein the one or more first lateral protrusions comprise a plurality of first lateral protrusions having a first gap disposed between adjacent ones of the first lateral protrusions, and wherein the one or more second lateral protrusions comprise a plurality of second lateral protrusions having a second gap disposed between adjacent ones of the second lateral protrusions.

Example 34. The method of any example disclosed herein, particularly example 33, further comprising stitching a laterally extending suture from one of the first gaps to one of the second gaps such that the laterally extending suture extends around the first suture and the second suture.

Example 35. The method of any example disclosed herein, particularly examples 30-33, wherein each of the one or more first lateral protrusions comprises a first aperture, and wherein each of the one or more second lateral protrusions comprises a second aperture.

Example 36. The method of any example disclosed herein, particularly example 35, further comprising stitching a laterally extending suture through the first aperture and the second aperture such that the laterally extending suture extends around the first suture and the second suture.

Example 37. The method of any example disclosed herein, particularly examples 30-36, wherein at least a portion of the paired first and second commissure tabs is covered with a flexible connector.

Example 38. The method of any example disclosed herein, particularly example 37, further comprising coupling first and second end portions of the flexible connector to the frame on an interior of the frame.

Example 39. The method of any example disclosed herein, particularly example 37, further comprising: wrapping a first portion the flexible connector over the one or more first lateral protrusions and the first suture; wrapping a second portion of the flexible connector over the one or more second lateral protrusion and the second suture; and coupling first and second end portions of the flexible connector to each other exterior of an exterior surface of the frame.

Example 40. A prosthetic heart valve according to any example herein, wherein the prosthetic heart valve is sterilized.

Example 41. A method of sterilizing any of the prosthetic heart valves described herein.

The features described herein with regard to any example can be combined with other features described in any one or more of the other examples, unless otherwise stated. For example, any one or more features of one commissure support structure can be combined with any one or more features of another commissure support structure. As another example, any one or more of the features of one prosthetic valve can be combined with any one or more features of another prosthetic valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a radially expandable and collapsible annular frame, the frame comprising a plurality of angularly spaced commissure support structures, each of the commissure support structures comprising axially oriented first and second struts, wherein each of the first and second struts comprises at least one laterally extending protrusion; and
a leaflet assembly comprising a plurality of leaflets each comprising commissures tabs on opposite sides of the leaflet, wherein a first commissure tab of each leaflet is paired with a second commissure tab of an adjacent leaflet to form a commissure, wherein each commissure extends radially between the first and second struts of one of the plurality of commissure support structures, wherein the first commissure tab is connected to the first strut with a first suture wrapped around the at least one protrusion of the first strut and the second commissure tab is connected to the second strut with a second suture wrapped around the at least one protrusion of the second strut.

2. The prosthetic heart valve of claim 1, wherein the first and second struts define a commissure window therebetween having a respective one of the commissures extending therethrough, and wherein the at least one protrusion of the first strut and the at least one protrusion of the second strut extend laterally outwardly relative to the commissure window.

3. The prosthetic heart valve of claim 2, wherein the at least one protrusion of the first strut is perpendicular relative to a first exterior edge of the commissure window, and wherein the at least one protrusion of the first strut is perpendicular relative to a second, opposing exterior edge of the commissure window.

4. The prosthetic heart valve of claim 1, further comprising a flexible connector extending over at least a portion of the first commissure tab and at least a portion of the second commissure tab, the flexible connector configured to prevent contact between the first commissure tab and the first strut and to prevent contact between the second commissure tab and the second strut wherein the first suture extends through the first commissure tab and the flexible connector, and wherein the second suture extending through the second commissure tab and the flexible connector.

5. The prosthetic heart valve of claim 4, wherein end portions of the flexible connector are each coupled to an interior of the frame via one or more sutures.

6. The prosthetic heart valve of claim 4, wherein the flexible connector wraps around side surfaces of the first and second struts and end portions of the flexible connector are coupled to each other on an exterior of the frame via one or more sutures.

7. The prosthetic heart valve of claim 1, wherein the at least one protrusion of the first strut comprises a plurality of first protrusions, and the at least one protrusion of the second strut comprises a plurality of second protrusions.

8. The prosthetic heart valve of claim 7, wherein adjacent ones of the first protrusions have first gaps disposed therebetween and adjacent ones of the second protrusions have second gaps disposed therebetween.

9. The prosthetic heart valve of claim 8, the first suture is woven through the first commissure tab and the first gaps, and wherein the second suture is woven through the second commissure tab and the second gaps.

10. A prosthetic heart valve comprising:
a radially expandable and collapsible annular frame, the frame comprising a commissure support structure including axially oriented first and second struts, wherein the first strut comprises a plurality of first laterally extending protrusions and the second strut comprises a plurality of second laterally extending protrusions; and
a leaflet assembly comprising a plurality of leaflets disposed within the frame, wherein a first commissure tab of a first one of the leaflets is paired with a second commissure tab of a second, adjacent one of the leaflets to form a commissure, wherein the first commissure tab is coupled to the first laterally extending protrusions via a first suture, and wherein the second commissure tab is coupled to the second laterally extending protrusions via a second suture.

11. The prosthetic heart valve of claim 10, wherein the first suture is woven between the first laterally extending protrusions, and wherein the second suture is woven between the second laterally extending protrusions.

12. The prosthetic heart valve of claim 10, wherein the first suture extends through the first commissure tab at first gaps between the first laterally extending protrusions, and wherein the second suture extends through the second commissure tab at second gaps between the second laterally extending protrusions.

13. The prosthetic heart valve of claim 12, wherein a lateral suture extends from one of the first gaps to one of the second gaps on an opposing side of the commissure support structure such that the lateral suture extends around the first and second sutures.

14. The prosthetic heart valve of claim 10, wherein the first laterally extending protrusions comprise first apertures and the second laterally extending protrusion comprise second apertures, and wherein a lateral suture extends from one of the first apertures to one of the second aperture on an opposing side of the commissure support structure such that the lateral suture extends around the first and second sutures.

15. The prosthetic heart valve of claim 10, wherein the first suture comprises a plurality of first stitches and the second suture comprises a plurality of second stitches, wherein each of the first stitches is looped over one of the first laterally extending protrusions and each of the second stitches is looped over one of the second laterally extending protrusions.

16. A method of assembling a prosthetic heart valve, the method comprising:
pairing a first commissure tab of a first leaflet with a second commissure tab of a second, adjacent leaflet;
inserting the paired first and second commissure tabs between first and second axially extending struts of a commissure support structure of an annular frame, the commissure support structure comprising one or more first lateral protrusions extending from the first strut and one or more second lateral protrusions extending from the second strut; and
coupling the first commissure tab to the one or more first lateral protrusions via a first suture and coupling the second commissure tab to the one or more second lateral protrusions via a second suture.

17. The method of claim 16, wherein the first suture is pre-attached to the first commissure tab and the second suture is pre-attached to the second commissure tab prior to inserting the first and second commissure tabs between the first and second axially extending struts, and wherein the coupling the first commissure tab comprises looping one or more stitches of the first suture over the one or more first lateral protrusions and the coupling the second commissure tab comprises looping one or more stitches of the second suture over the one or more second lateral protrusions.

18. The method of claim 16, wherein the coupling the first commissure tab comprises aligning the first commissure tab with the one or more first lateral protrusions and stitching the first suture through the first commissure tab and around the one or more first lateral protrusions, and wherein the coupling the second commissure tab comprises aligning the second commissure tab with the one or more second lateral protrusions and stitching the second suture through the second commissure tab and around the one or more second lateral protrusions.

19. The method of claim 16, wherein the one or more first lateral protrusions comprise a plurality of first lateral protrusions having a first gap disposed between adjacent ones of the first lateral protrusions, and wherein the one or more second lateral protrusions comprise a plurality of second lateral protrusions having a second gap disposed between adjacent ones of the second lateral protrusions, the method further comprising stitching a laterally extending from one of the first gaps to one of the second gaps such that the laterally extending suture extends around the first suture and the second suture.

20. The method of claim 16, wherein each of the one or more first lateral protrusions comprises a first aperture, and wherein each of the one or more second lateral protrusions comprises a second aperture, the method further comprising stitching a laterally extending suture through the first aperture and the second aperture such that the laterally extending suture extends around the first suture and the second suture.
